(19) United States
(12) Patent Application Publication
(10) Pub. No.: US 2014/0294745 A1
(43) Pub. Date: Oct. 2, 2014

(54) CORAL EXTRACT FOR SKIN WHITENING, MOISTURE RETENTION, ELASTICITY IMPROVEMENT, ANTI-INFLAMMATION AND OCCLUSION OF WOUNDS AND EXTRACTING METHOD THEREOF

(71) Applicant: UNI-PRESIDENT BIOTECH CO., LTD., Tainan City (TW)

(72) Inventors: PING-JYUN SUNG, TAINAN CITY (TW); WEI-HSIEN WANG, TAINAN CITY (TW); ZHI-HONG WEN, TAINAN CITY (TW); YI-SHAN WEN, TAINAN CITY (TW); TAI-TING CHOU, TAINAN CITY (TW); YIN-PIN CHENG, TAINAN CITY (TW); CHIH-CHIEH KUO, TAINAN CITY (TW)

(73) Assignee: UNI-PRESIDENT BIOTECH CO., LTD., Tainan city (TW)

(21) Appl. No.: 14/160,727

(22) Filed: Jan. 22, 2014

(30) Foreign Application Priority Data
Apr. 1, 2013 (TW) 102111780

(51) Int. Cl. A61K 8/98; A61Q 19/02; A61Q 19/00; A61K 35/56

(52) U.S. Cl. CPC A61K 8/987; A61K 35/614; A61Q 19/02; A61Q 19/007
USPC 424/62; 424/520

(57) ABSTRACT

A coral extract for skin whitening, moisture retention, elasticity improvement, anti-inflammation and occlusion of wounds and its extracting method is provided. The coral extract is extracted from Briareum excavatum. The extracting method comprises: extracting with organic solvents, partitioning with water and ethyl acetate and separating via column chromatography. The coral extract can be applied in the field of skin whitening, moisture retention, elasticity improvement, anti-inflammation and occlusion of wounds.

### BACKGROUND OF THE INVENTION

1. Field of the Invention

The invention relates to a coral extract, extracting method and purpose, and more particularly to a Briareum excavatum extract, wherein the extract has effects of enhancing moisture retention, elasticity, inhibiting tyrosinase activity, reducing melanin for skin and slowing down the infiltration of inflammatory cells and controlling wound areas.

2. Description of the Related Art

Bio-synthesis path and enzyme system of secondary metabolites produced by marine organisms are obvious difference due to specificity of marine environment by comparing with geobion. Therefore, marine organisms usually produces marine secondary metabolites having novel chemical structures and diverse biological activity as well as different structure features in comparison with geobion.

Coral belongs Coelenterata and has more than 9000 species and is one of the most common creatures. Coral is about 22.4% marine species and a marine living resource capable of being utilized in mass. Gorgonian is so-called pectin, sea whip and gorgonia and a major branch in coral animals. With respect to the research of coral chemical compound, prostaglandin precursors, which has abundant unique structure and stronger physiological activity and has been found by Weinheiner et al. (U.S.A., 1969) from gorgonian, attracts numerous natural product chemists to focus on the research marine species from geobion. These marine natural products have wide biological activity and can mainly affect neural systems, cardiovascular systems, immune systems, etc., and many of them have obvious antitumor activity.

Recently, the research for gorgonian is still in full swing. For example, many briarane type diterpenes having activity are found from gorgonian of Briareum genus. However, the source of wild coral is restricted so that some researchers try to artificial culture Briareum excavatum of alcyonacena in laboratories. Unfortunately, none of literatures or patents has showed that extracted products or purified compounds capable of restraining tyrosinase activity, accelerating wound healing and resisting inflammation can be obtained from Briareum excavatum.

### SUMMARY OF THE INVENTION

Therefore, upon the foregoing problems of prior art, it is an objective of the present invention to provide a coral extract obtained by extracting Briareum excavatum and having efficacies of moisture retention, elasticity improvement, melanin reduction as well as slow down of inflammation cell infiltration and wound area control for skins. The coral extract is obtained by the following steps:
a. extract dried Briaerum excavatum samples from an organic mixture to obtain an organic layer initial extract, wherein the organic mixture constitutes C₁-C₄ alcohol and C₁-C₄ chlorinated alkanes in equal proprotions;
b. distribute and extract the organic layer initial extract through water and low class ester to obtain a low class ester crude extract, the low class ester crude extract containing a 5-50% coral active constituent.

Another objective of the invention is to provide an extracting method for coral extract, and the method is simple and easily produced in mass, and the method comprises the following steps:
a. extract dried Briareum excavatum samples through an organic mixture to obtain an organic layer initial extract, wherein the organic mixture contains C₁-C₄ alcohol and C₁-C₄ chlorinated alkanes in equal proprotions;
b. distribute and extract the organic layer initial extract through water and low class ester to obtain a low class ester crude extract, the low class ester crude extract containing a 5-50% coral active constituent.

In a preferred embodiment, the step b of the low class ester extract is further purified by column chromatography, and the low class ester crude extract is eluted by an eluent to obtain an eluted substance, wherein gradient elution of the eluent is provided by a first mixed solvent of hexane/ethyl acetate and a second mixed solvent of ethyl acetate/methanol.

In a preferred embodiment, the C₁-C₄ alcohol of the step a is methanol but also includes ethanol, propanol and the like. In another preferred embodiment, the C₁-C₄ chlorinated alkanes of the step a is methylene chloride but also includes chlorinated alkanes such as methylene chloride, chloroform and the like.

In a preferred embodiment, the low class ester of the step b is ethyl acetate.

In another embodiment, a solvent gradient of hexane within the first mixed solvent is 0% to 99%. In another word, hexane: ethyl acetate can form the gradient at any ratio from 99:1 to 0:100. For example, it can be any ratio among 99:1 to 93:7, 90:10 to 70:30, 60:40 to 50:50, 10:90 to 0:100.

In another embodiment, the solvent gradient of ethyl acetate is 0% to 90% in the mixed solvent of ethyl acetate/methanol. In another word, ethyl acetate: methanol can form the gradient at any ratio from 90:10 to 0:100. For example, it can be any ratio from 90:10 to 50:50, 40:60 to 0:100.

In a preferred embodiment, the coral active constituent is obtained by eluting the low class ester crude extract through the first mixed solvent, wherein gradient elution of the first mixed solvent is that: hexane: ethyl acetate: ethyl acetate is fraction of 90:10 to 70:30. Preferably, the coral active constituent is 5 to 30% proportion of the low class ester crude extract after elution.

In a preferred embodiment, the coral active constituent is obtained by eluting the low class ester crude extract through the first mixed solvent, wherein gradient elution of the first mixed solvent is that: hexane: ethyl acetate is fraction of 60:40 to 50:50. Preferably, the coral active constituent is 20 to 50% proportion of the low class ester crude extract after elution.

Another objective of the invention is to provide a purpose of coral extract for imposing the coral extract on live skin to whiten skin.

Another objective of the invention is to provide a purpose of coral extract for imposing the coral extract on live skin to perform moisture retention.

Another objective of the invention is to provide a purpose of coral extract for imposing the coral extract on live skin to improve skin elasticity.

Another objective of the invention is to provide a purpose of coral extract for imposing the coral extract on live skin to resist inflammation.

A further objective of the invention is to provide a purpose of coral extract for imposing the coral extract on live skin to heal wounds.

A further objective of the invention is to provide a skincare product comprising the coral extract, wherein the coral extract has 0.00001 to 10% weight percentage

### BRIEF DESCRIPTION OF THE DRAWINGS

The detailed structure, operating principle and effects of the present invention will now be described in more details hereinafter with reference to the accompanying drawings that show various embodiments of the invention as follows.

FIG. 1 is a crude extraction and separation steps of active constituent from breeding type Briareum excavatum;

FIG. 2 is a ¹H-NMR spectrum of BP1;

FIG. 3 is a ¹H-NMR spectrum of BP2;

FIG. 4 is a ¹H-NMR spectrum of BP3;

FIG. 5 is a ¹H-NMR spectrum of BP4;

FIG. 6 is a ¹H-NMR spectrum of BP5;

FIG. 7 is a ¹H-NMR spectrum of BP6;

FIG. 8 is a ¹H-NMR spectrum of BP7

FIG. 9 is an animal skin allergy testing result performed by each crude extract from Briareum excavatum;

FIG. 10 is an experimental result of tyrosinase activity assay in vitro;

FIG. 11 is an experimental result of melanin containing assay in vitro;

FIG. 12 is an experimental result of BP2 whitening activity assay in vivo;

FIG. 13 is an experimental result of BP3 whitening activity assay in vivo;

FIG. 14 is an experimental result of wound healing assay in vitro performed by fibroblast: NIH/3T3 of rats, and (A)(B) are the control group that is not supplied with test substances; and (C)(D) BP2 are a low dose group, and its concentration is 10 μg/ml; and (E)(F)BP2 are a high dose group, and its concentration is 20 μg/ml; and (G)(H)BP3 are a low dose group, and its concentration is 10 μg/ml; and (I)(J)BP3 are a high dose group, and its concentration is 20 μg/ml; and (K) is statistics of each group;

FIG. 15 is an experimental result of wound healing assay in vitro performed by human umbilical vein endothelial cell line: EA.hy926, (A)(B) are a control group and not supplied with test substances; and (C)(D)BP2 are a low dose group, and its concentration is 10 μg/ml; and (E)(F)BP2 are a high dose group, and its concentration is 20 μg/ml; and (G)(H)BP3 are a low dose group, and its concentration is 10 μg/ml; and (I)(J)BP3 are a high dose group, and its concentration is 20 μg/ml; and (K) is statistics of each group;

FIG. 16 is an experimental result of wound healing assay healed by 3 mg/0.2 ml BP2, BP3, and pure cream;

FIG. 17 is an experimental result of wound healing assay healed by 40 μg/0.2 ml BP2, BP3, crude extract and pure cream;

FIG. 18 is a histopathological slice of burned skin tissue of the Wistar rat stained by HE staining (5×), and figure A is the control group, figure B is the burned group, figure C is the BP2 group, figure D is the BP3 group, and the concentration of provided natural substance is 3 mg/0.2 ml;

FIG. 19 is a histopathological slice of burned skin tissue of the Wistar rat stained by HE staining (5×), and figure A is the control group, figure B is the burned group, figure C is the BP2 group, figure D is the BP3 group, figure E is the crude extract group, and the concentration of provided natural substance is 40 μg/0.2 ml;

FIG. 20 is a moisture retention comparison between three prototype skincare products and cream matrix; and

FIG. 21 is an elasticity comparison among three prototype skincare products, a blank group and cream matrix.

### DETAILED DESCRIPTION OF THE PREFERRED EMBODIMENTS

The technical content of the present invention will become apparent by the detailed description of the following embodiments and the illustration of related drawings as follows.

### PREPARED EXAMPLE 1

### Briareum excaatum Extracted by Organic Solvent

Bio-sample Briareum excavatum (1021.49 g, wet weight) mass-bred in a 0.6 tons breeding water tank is carried out freezing-dry, and dried coral tissues are pulverized to obtain dried weight 417.75 g. Afterward the extraction is performed at mixed ratio 1:1 of organic solvent methanol/methylene dichloride. Until added organic solvent is clear through many times of repeated extractions, organic layer initial extract is obtained. The initial extract then is carried out vacuum concentration after filtration so that obtained crude extract is distributed and extracted by water and ethyl acetate, and the crude extract (15.75 g) in ethyl acetate layer, which has been carried out distribution extraction and vacuum concentration, is further performed with the following separation process to obtain BP2 (2.44 g, sharing about 15.5% of crude extract weight) and BP3 (5.6 g, sharing about 35.6% of crude extract weight).

### PREPARED EXAMPLE 2

### Separation Process for Crude Extract

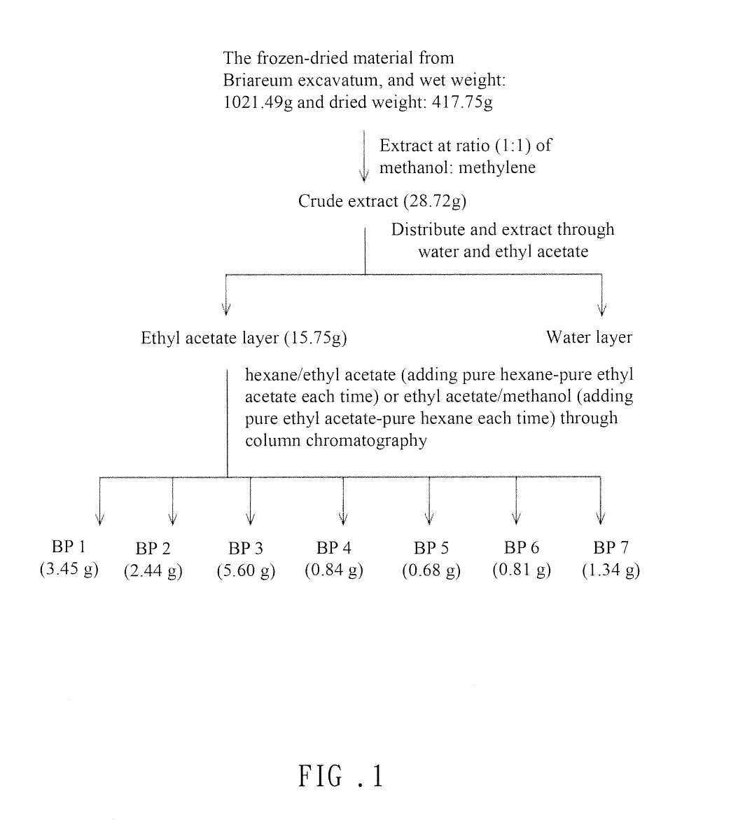

With reference to FIG. 1, the ethyl acetate layer is carried out initial separation by utilizing column chromatography. A selected filler is silicon gel (Merck, 230-400 mesh), and a mixed solvent from hexane/ethyl acetate and ethyl acetate/methanol taken as eluent is used for gradient elution, and seven fraction is divided, wherein each fraction's elution condition is shown as the following:

Fraction 1 (BP1): Hexane:EtOAC (hexane: ethyl acetate) 99:1-93:7,

Fraction 2 (BP2): Hexane:EtOAC (hexane: ethyl acetate) 90:10-70:30.

Fraction 3 (BP3): Hexane:EtOAC (hexane: ethyl acetate) 60:40-50:50.

Fraction 4 (BP4): Hexane:EtOAC (hexane: ethyl acetate) 40:60-20:80.

Fraction 5 (BP5): Hexane:EtOAC (hexane: ethyl acetate) 10:90-0:100.

Fraction 6 (BP6): EtOAC:MeOH (ethyl acetate: methanol) 90:10-50:50.

Fraction 7 (BP7): EtOAC:MeOH (ethyl acetate: methanol) 40:60-0:100.

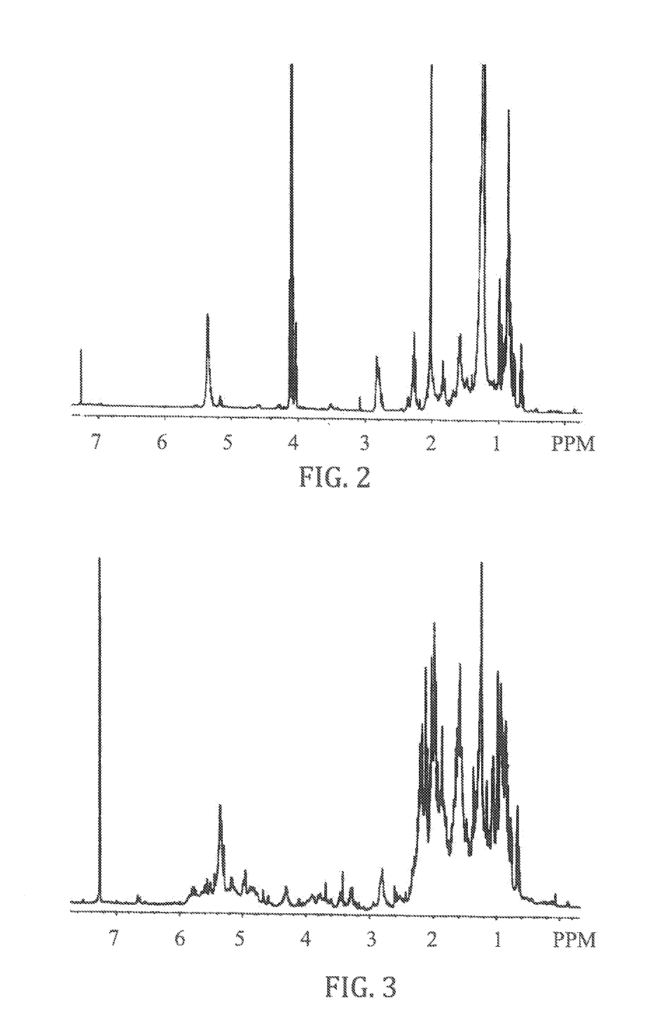

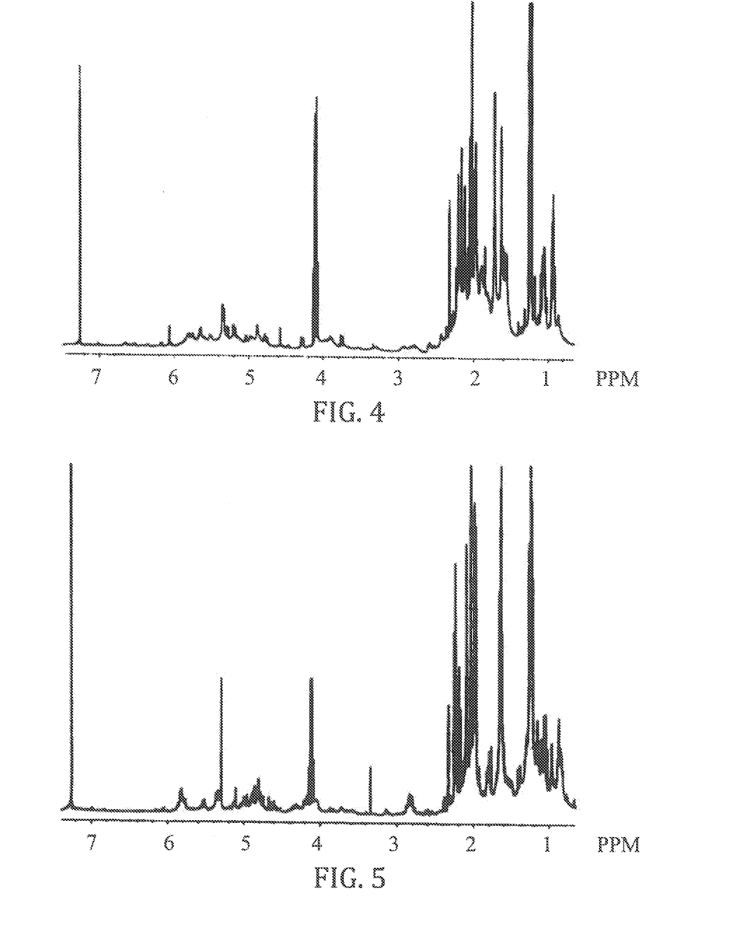

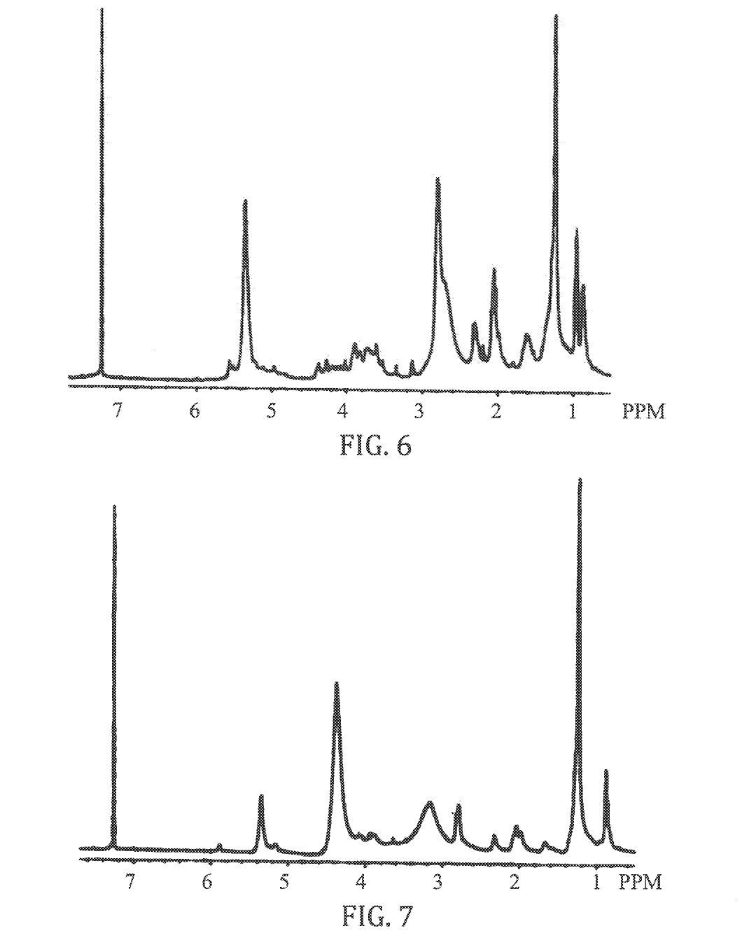

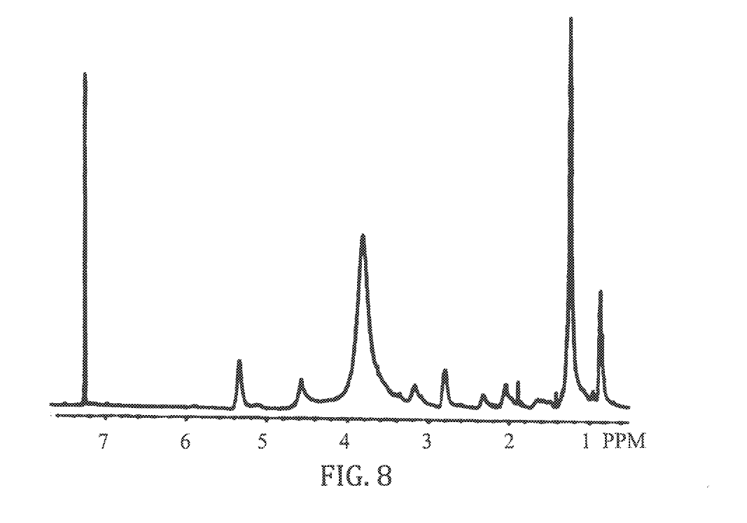

Afterward each fraction, which has been processed with vacuum concentration, is detected by a nuclear magnetic resonance to obtain a signal of ¹H-NMR spectra (with reference to FIG. 2 to FIG. 8). The spectra is taken as a basis of determining the existence of marker constituents and will be further treated as the basis for bioassays.

### TEST EXAMPLE 1

### Activity Test for Anti-Inflammatory In Vitro

1. Natural Compound Screening and Cell Strain Usage

In connection with the crude extract that has been processed by steps of gradient elution, raw cells strain RAW 264.7 is induced with inflammation in vitro mode to perform screening by using lipopolysaccharide. The number of raw cells RAW 264.7 in a culture dish having 6 centimeters wide is controlled at 3×10⁶. The crude extract is firstly provided, and cells then are collected after supplying lipopolysaccharide (LPS) for 16-18 hours.

2. Western Bolt Analysis for Protein Expression Level

4% phosphate buffered saline (PBS, 137 mM NaCl, 2.68 mM KCl, 10 mM Na₂HPO₄, 1.76 mM KH₂PO₄, pH=7.2) is collected in a 1.5 ml centrifuge tube, and supernatant liquid is removed after centrifuging at 3000 rpm for 8 minutes, and lysis buffer 200 μl (50 Mm Tris, pH 7.5, 150 mM NaCl, 1% TritonX-100, 0.1 mM EDTA, 0.1 Mm EGTA, 100 μg/ml phenylmethylsulfonyl fluoride, 1 μg/ml Aprotinin, 20 mM NaF, 0.2 mM Na₃VO₄) at 4 degrees Celsius is added to break cell membranes. At 4 degrees Celsius, the centrifuging is performed at 14,000 rpm for thirty minutes, and supernatant liquid is taken out, and method provided by Lowry et al., 1951 is further simulated to perform quantitative protein.

The absorbance of the supernatant liquid is analyzed by Bio-Rad DC protein assay kit (Hercules, Calif., U.S.A.) and an enzyme reader (Thermo Electron Corporation, U.S.A.) to measure protein quantity of each sample. A sample buffer (2% SDS, 10% glycerol, 0.1% bromophenol bule, 10% 2-mercaptoethanol, 50 mM Tris, Bio-Red Laboratories, Inc.) sharing one third sample total volume is taken after performing calibration, and, with 135 milliampere, protein is separated at 80 voltages by using 7%, 10% SDS-PAGE to transfer protein from SDS-PAGE to a PVDF film (0.45 mm pore size, lmmobilon-P, Millipore, Bedford, Mass., USA) overnight.

The transfered PVDF film is covered by TTBS solution (Tris-Tween buffer saline) (Tris-HCl 20 mM, NaCl 137 mH, pH 7.4, 0.1% Tween 20) containing 5% skim milk powder at room temperature for 40 minutes, then is reacted with a primary antibody affected by inducible nitric oxide synthase and cyclooxygenase-2 (COX-2) at 1:1000 dilution ratio for two hours at room temperature. Subsequently, the film is further rinsed by TTBS solution three times. Next, at room temperature, the film is reacted for one hour thirty minutes through HRP-conjugated anti-rabbit IgG antibodies (1:2000) to form secondary antibodies. When the secondary antibodies are finished, the film is rinsed by TTBS solution for three times. Finally, the PVDF film is reacted with developer (Immobilon Western Chemiluminesccent HRP Substrate, Millipore Corporation, Billerica, Mass. 01821 U.S.A.), and luminescent reaction is detected by an image analysis processing facility (UVP Biospectrum AC system, UVP Inc., U.S.A.) to record protein expression, and computer analysis software (VisionWorks LS Acquisition and analysis software, copyright 2007, LLC, U.S.A.) is used to detect and calculate relative quantity, and defined the protein expression level of the lipopolysaccharides added individually group is 100%. Finally, α-actin is taken as the inner control group.

By comparing with the achievement of crude extracts of different fraction layers for anti-inflammatory in vitro, its result is further taken as the reference for vivo experimental mode and other in vitro experimental modes.

3. Used Target Antibodies

(1) Inducible nitric oxide synthase (BD Pharmingen, San Diego, Calif., U.S.A.: catalog no. 6103322: polyclonal antibody), its dilution ratio is 1:1000.

(2) Cyclooxygenase-2 (COX-2) (Cayman Chemical, U.S.A.; catalog no. 160106; polyclonal antibody), its dilution ratio is 1:1000.

(3) β-actin (sigma, St. Louis, Mo., U.S.A.; catalog no. A5316-2ML; monoclonal antibody), its dilution ratio is 1:2000.

4. Test Result

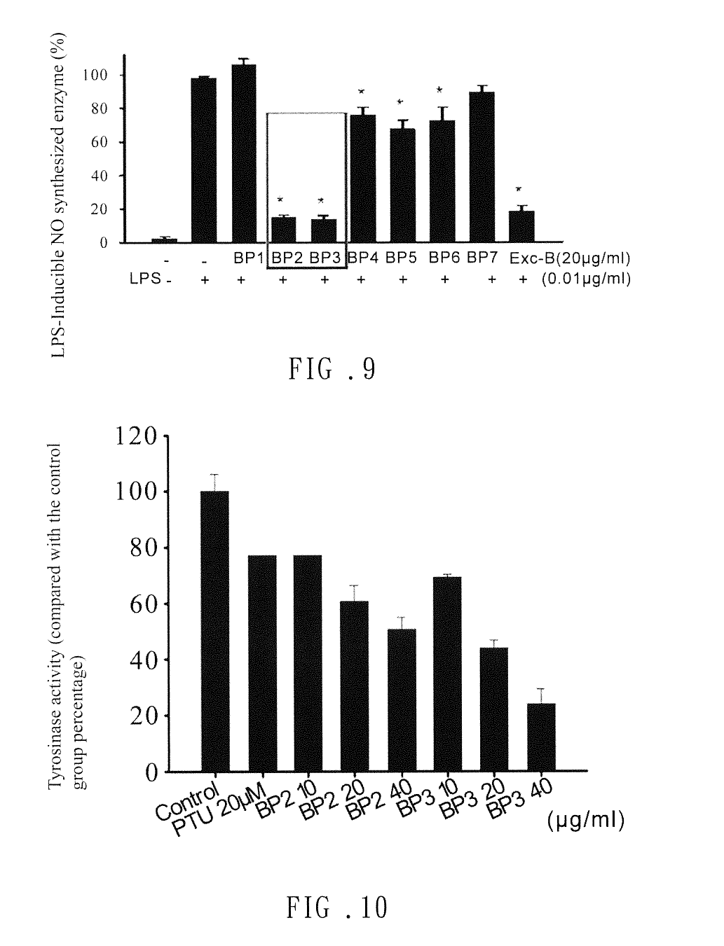

With reference to FIG. 9, by comparing with crude extracts of different fraction layers for anti-inflammatory in vitro, 20 μg/ml BP2 (fraction 2) and BP3 (fraction 3) have extremely outstanding performance in anti-inflammatory in vitro.

### TEST EXAMPLE2

### Whitening Activity Analysis

A. Tyrosinase Activity Assay In Vitro

1. Test Procedure

The test procedure is referred to Wang's research paper (2010), and its whole content is cited into reference information of the embodiment. B16-F10 is planted into holes of a 24 well plate. Each well is planted 1×10⁵ cells. After the plate then is placed in a cultivation chest having 5% CO₂ at 37 degrees Celsius for twenty-four hours, original cell culture medium is drained away, and a cell culture medium containing crude extracts is added. The plate then is placed in the cultivation chest for forty eight hours. When administration of medicine is finished, original cell culture medium is drained away, and the cells are rinsed by adding 200 μl PBS for one time. Afterward 100 μl trypsin is added to affect for one to two minutes so that cells come off the bottom. 200 μl cell culture medium is further added to neutralize trypsin reaction, and the culture medium containing cells is placed in a micro-centrifuge tube to centrifugally rotate at 3000 rpm for five minutes in order to drain supernatant liquid away. 200 μl PBS is added to rinse for one time, and centrifugal rotation at 3000 rpm is performed for five minutes to drain supernatant liquid away, and 100 μl triton X-100/PBS is added to uniformly mix the mixture to crumble cells. Next, centrifugal rotation at 10000 rpm and 4 degrees Celsius is performed for ten minutes. Supernatant liquid is taken to perform protein quantitation. The protein under the same concentration is reacted with the mixing of L-dopa (2 mg/ml) and equivalent volume supernatant liquid for one hour, and the enzyme reader is used to measure the absorbance having 475 nm wavelengths to carry out statistics.

2. Test Result

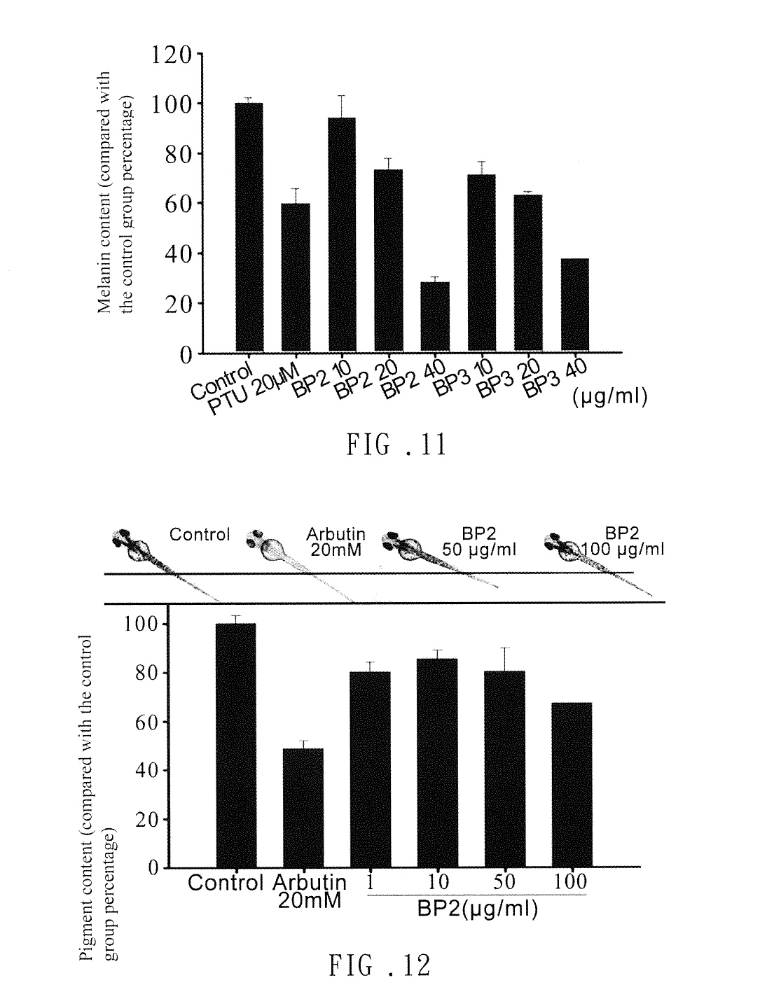

With reference to FIG. 10, the test result is that BP2 and BP3 having different concentrations are added in melanoma cells to perform tyrosinase activity assay after forty-eight hours, wherein X-axis is each concentration of medicines, and Y-axis is in vitro tyrosinase activity/melanin content. If produced products are much more, the catalytic intensity is powerful. In the result, under the same protein concentration, the groups of providing BP2 and BP3 can restrain tyrosinase activity, the tendency of dose-dependent is further provided in accordance with raised concentration.

B. In Vitro Melanin Content Assay

1. Test Procedure

The test procedure is referred to Wang's research paper (2010), and its whole content is cited into reference information of the embodiment. B16-F10 is planted into pores of a 24 well plate. Each well is planted with 1×10⁵ cells. After the plate then is placed in a cultivation chest having 5% CO₂ at 37 degrees Celsius for twenty-four hours, original cell culture medium is drained away, and a cell culture medium containing crude extracts is added. After the plate then is placed in the cultivation chest for forty eight hours, 200 μl PBS is used to rinse it for one time. Afterward 100 μl trypsin is added to affect for one-two minutes so that cells come off the plate bottom. 200 μl cell culture medium is further added to neutralize trypsin reaction, and the culture medium containing cells is placed in a micro-centrifuge tube to centrifugally rotate at 3000 rpm for five minutes. After draining supernatant liquid away, at 80 degrees Celsius, 100 μl 1N NaOH is added to react for one hour. The enzyme reader is used to measure the absorbance having 405 nm wavelengths to carry out statistics.

2. Test Result

With reference to FIG. 11, the test result is that BP2 and BP3 having different concentrations are added in melanoma cells to perform melanin assay after forty-eight hours. After cells are added with sodium hydroxide and further heated/melted, a spectrophotometer is utilized to measure absorbance having 490 nm wavelength. The melanin content is also higher when the absorbance is higher. The result matches with tyrosinase activity. Since the concentration of medicines rises, tyrosinase activity is reduced to reach more significance, and the expression of melanin is also decreased accordingly.

C. Mushroom Tyrosinase Inhibition Capability Analysis

1. Test Procedure

In a 96 well plate, 70 μl PBS, 20 μl specimen solution having different concentrations is added into each pore, and the mixture is uniformly mixed. Next, 10 μl 12U tyrosinase is added to place the plate in the cultivation chest at 37 degrees Celsius to react for thirty minutes. 10 μl 15 mM L-dopa is further added, and after continuously placing the plate in the cultivation chest to react for thirty minutes, the variance of absorbance is measured at 492 nm wavelengths. By taking H₂O or 50% EtOH as the control group, the anti-oxidation rate of the object to be tested is obtained by inhibition of tyrosinase % (IT %) as the following formula. When peroxidation rate of lipid inhibition is higher, antioxidative function is stronger.

Inhibition of tyrosinase %, IT %=[1−(absorbance of sample at 492 nm)/(absorbance of the control group that is not added with samples at 492 nm)]×100.

2. Test Result

Tyrosinase is a rate deciding factor forming melanin. If the object to be tested can inhibit tyrosinase activity, production of dopaquinone type compound can be reduced. The capability for the object to be tested inhibiting tyrosinase activity can be calculated in high/low of absorbance, thereby further restraining generation of melanin. The test result is that 500 μg/ml or 1000 μg/ml concentration of BP2 and BP3 have obvious feature of inhibiting tyrosinase activity, and its test result can refer to the following table 1:

Mushroom tyrosinase inhibition capability assay (%)

| | |
|---|---|
| Negative control group (blank) | 0.0 |
| Positive control group (vitamin C, 100 μM) | 38.0 ± 0.4 |

Mushroom tyrosinase inhibition capability analysis

| Compound (μg/ml) | BP-2 | BP-3 |
|---|---|---|
| 500.0 | 21.2 ± 1.3 | 11.8 ± 1.4 |
| 1000.0 | 46.5 ± 4.2 | 29.7 ± 1.2 |

D. Whitening Activity Analysis In Vivo

1. Test Procedure

a. Brood Fish Breeding

The test uses zebra fishes (AB strain Danio rerio), in which fish edge is above four months, as test brood fishes, and the fishes are bred in an acrylic water tank having a filter and a circulation system. Water temperature is controlled at 28.5 degrees Celsius, and light-dark cycle is respectively controlled at fourteen and ten hours.

b. Medicine Preparation

The medicine to be tested must be dissolved in 100% DMSO solution, and finally concentration must also concern DMSO and medicine concentration.

c. Medicine Provision

Embryos that have been fertilized for nine hours is taken and injected into a 96 well culture dish, and three embryos and 100 μl Hank's buffer are disposed in each well. 100 μl taken from the prepared medicine is injected into each well, and after adequately and uniformly mixing the mixture, an orifice plate is covered to avoid draining water content away to change the concentration. The plate then is placed in a low-temperature illumination cultivation chest (Model RI-80, Firstek, Taiwan), and light-dark cycle is controlled at fourteen and ten hours (the cycle must equal the light cycle of brood fishes), and the temperature is retained at 28.5 degrees Celsius, and the mixture is continuously immersed and supplied with medicines for forty-eight hours.

d. Fish Image Capturing

Larval fishes, which have been supplied with medicine for forty-eight hours, is taken out and sequentially placed on a cavity of a hollow-ground slice (Micro Scientific Laboratories, Inc., U.S.A.) after using anesthetic agent to anesthetize them. The fishes are fastened by using 1% methyl cellulose (Sigma, U.S.A.), and a stereomicroscope (Z16 APO, Leica, Heerbrugg, Switzerland) then is used to observe the fishes, and an image capture system (idea SPOT, Diagnostic instruments Inc., U.S.A.) and its control software (SPOT software VERSION 4.6, Diagnistic instruments Inc., U.S.A.) are incorporated to obtain fish images. The exposure time of the test is set at 3.372 msec.

e. Data Processing

Obtained fish images are opened by image processing software (Image J 1.43 g; National Institute of Health, Bethesda, Md., U.S.A.), and under ranges where the minimum threshold is set at 0, and where the maximum threshold is set at 85, melanin of each group of zebra fishes is performed with quantitative analysis.

2. Test Result

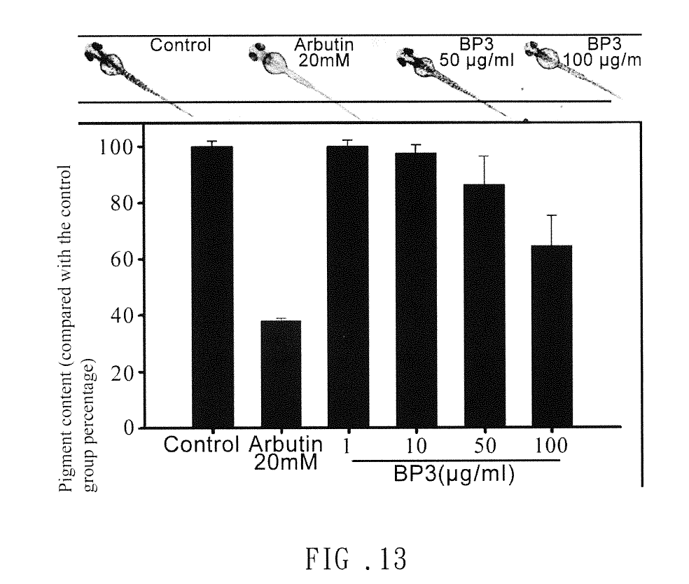

With reference to FIG. 12 and FIG. 13, melanin of zebra fishes is observed, and melanin content only remains at 50% when arbutin is at 20 mM (it approximately equals 54446 μg/ml). While concentrations of BP2 and BP3 are higher, melanin content gradually declines to 70%. Its concentration is one five-hundredth times' arbutin although the whitening degree of BP2 and BP3 in the test result is not better than the positive control group. Therefore, two crude extracts have effective compounds providing excellent whitening potential.

### TEST EXAMPLE 3

### Wound Healing Activity Assay

A. Wound Healing Activity Assay

1. Test Procedure

The research of Rodriguez (2005-2010) with respect to in vitro wound healing assay of fibroblast, NIH/3T3 and human umbilical vein cell line, EA.hy926 is referred, and its whole is cited into reference information of the embodiment. Cells are planted into pores of a 12 well culture plate, and 5×10⁵ cells are planted in each well. After the plate is placed into the cultivation chest having 5% CO₂ at 37 degrees Celsius for twenty four hours, the original cell culture medium is drained away, and a horizontal scar is drawn by utilizing 200 μl tip. Next, 200 μl PBS is added to rinse it, and photographing is taken after adding the cell culture medium containing crude extract. The same region is photographed with pictures after the plate then is placed into the cultivation chest having 5% CO₂ at 37 degrees Celsius for twenty four hours. The picture is analyzed by image analysis software (TScratch version 1.0) to perform statistics after obtaining data.

2. Test Result

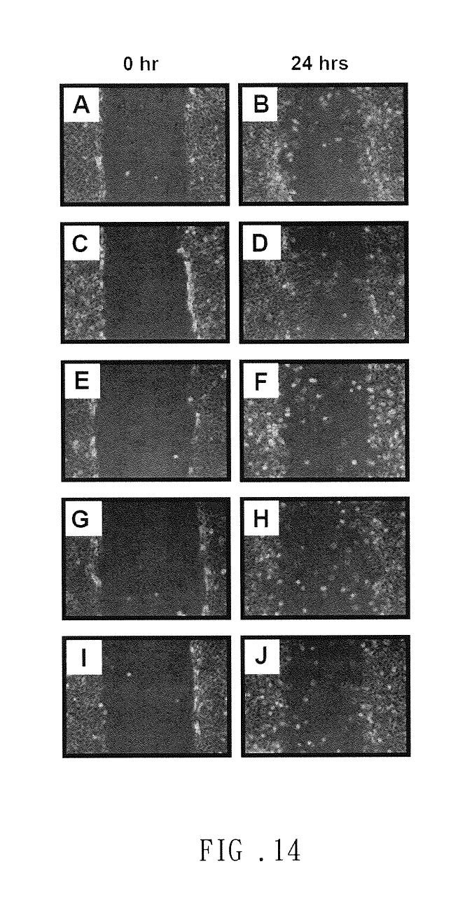

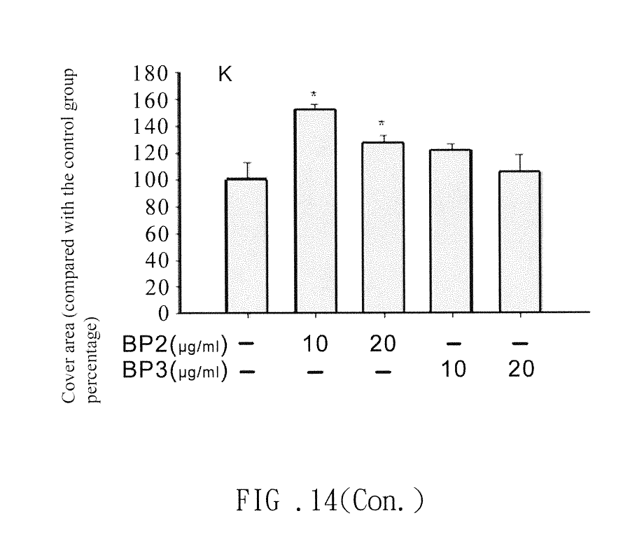

With reference to FIG. 14, the in vitro wound healing test result is performed by fibroblasts: NIH/3T3 of mice. (A)(B) is the control group in the figure and not supplied with test substances; and (C)(D)BP2 is a low dose group, and its concentration is 10 μg/ml; and (E)(F) BP2 is a high dose group, and its concentration is 20 μg/ml; and (G)(H) BP3 is a low dose group, and its concentration is 10 μg/ml; and (I)(J) BP3 is a high dose group, and its concentration is 20 μg/ml. SPOT Advanced software is used to calculate the area of scratch portion within the images. Statistics is performed as shown in the figure (K) after obtaining the area of the scratch covered by cells at twentieth four hour. The result shows that the cell cover areas provided with the low dose group of BP2 and the high dose group of BP2 are improved by comparing with the control group.

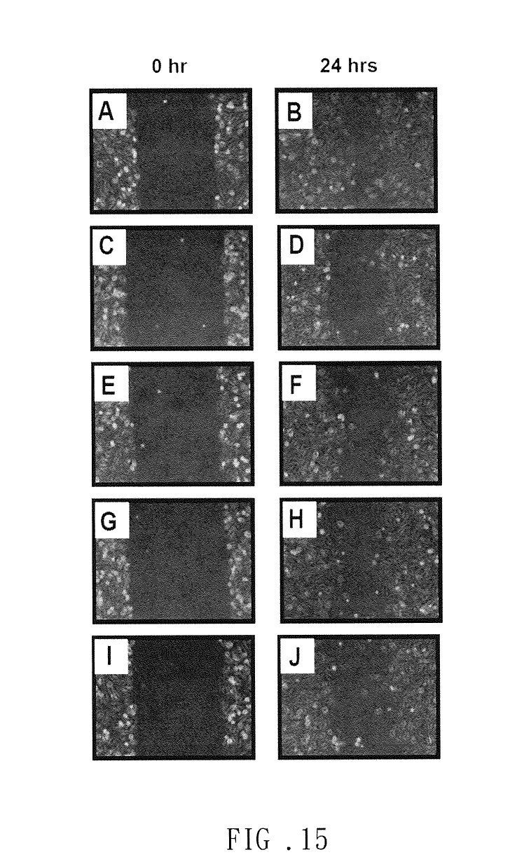

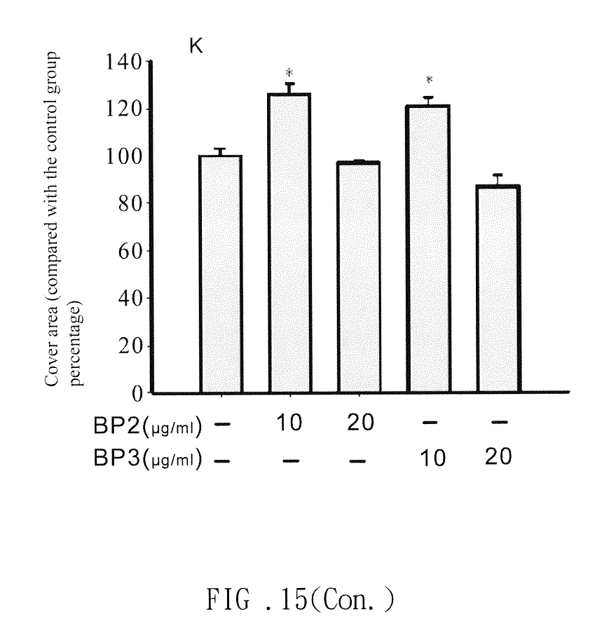

With reference to FIG. 15, the in vitro wound healing test result is performed by human umbilical vein endothelial cell line: EA. Hy926. In the figure, (A)(B) is the control group and not provided with test substances. (C)(D) BP2 is the low dose group, and its concentration is 10 μg/ml; and (E)(F) BP2 is the high dose group, and its concentration is 20 μg/ml. (G)(H) BP3 is a low dose group, and its concentration is 10 μg/ml; and (I)(J) BP3 is a high dose group, and its concentration is 20 μg/ml. Further, the area of scratch portion is calculated by using SPOT Advanced software, and statistics is performed after obtaining the area of scratch covered by cells at twentieth four hour. The result shows that the cell cover areas provided with the low dose group of BP2 and BP3 are obviously increased by comparing with the control group.

B. Wound Healing Activity Assay In Vivo

1. Wound Healing Test:

a. Experimental animal provision: the experimental animal is Wistar rat male rats having 400-450 g that are bred in the Marin Biotechnology and Resource's animal facility, National Sun Yat-sen University, Taiwan. The light cycle is that twelve hours' light irradiation and twelve hours' darkness. The Wistar rat can freely drink water and eat food, and an air condition system controls temperature and humidity of the breeding environment to retain the environmental temperature at 23 degrees Celsius.

b. Burned wound generation: in vivo wound healing assay is referred to the research of Huang (2008)11), and its whole is cited into reference information of the embodiment. After randomly dividing the Wistar rats into groups, an animal shaving machine is used to remove hairs of backs of the Wistar rats under anesthesia of 2.5% isoflurane (anesthesia unit: Isotec 4, Ohmeda), razors are further used to cleanly remove surplus hairs of the backs of the Wistar rats. Next, after using alcohol to perform disinfection, surgical blades are used to remove full-layered skins of the backs of the Wistar rats at two centimeter diameter. The healing group is immediately provided with medicine according to experimental design while the wound group is not healed. Each Wistar rat is individually bred after completing surgery.

c. Natural substance provision for wound: a natural substance (the natural substance is mixed by coral crude extract and a blank-based agent provided by an applicant, wherein the detail compound of the blank-based agent is referred as the following) is obtained at the same time every day to uniformly put on the wounds. Before healing the wounds every time, the wounds are firstly rinsed by sterile saline to clean surplus natural substance last night, and simultaneously, foreign matters are removed. The wounds then are uniformly imposed with medicine after using sterile cotton swab to dry physiological saline.

d. Wound observation and area calculation: with respect to burns according to experimental design date, the Wistar rat is placed on the copy stand after performing anesthesia. A series of pictures is taken by a digital camera (Coolpix P6000, Nikon, Japan) under the same condition (the diaphragm is 7.2, and the camera shutter is 1/60). Digital image capture system software (Diagnostic Instruments, Inc., Sterling Heights, Mich., U.S.A.) is used to analyze the captured wound picture to calculate the wound area. Data presenting portions of the wound area at each observation point are respectively presented at percentage of the wound area relative to day zero. At the same time, the weight of the Wistar rat is measured, and the Wistar rat is observed to find out any obvious strange behavior.

2. Test Result

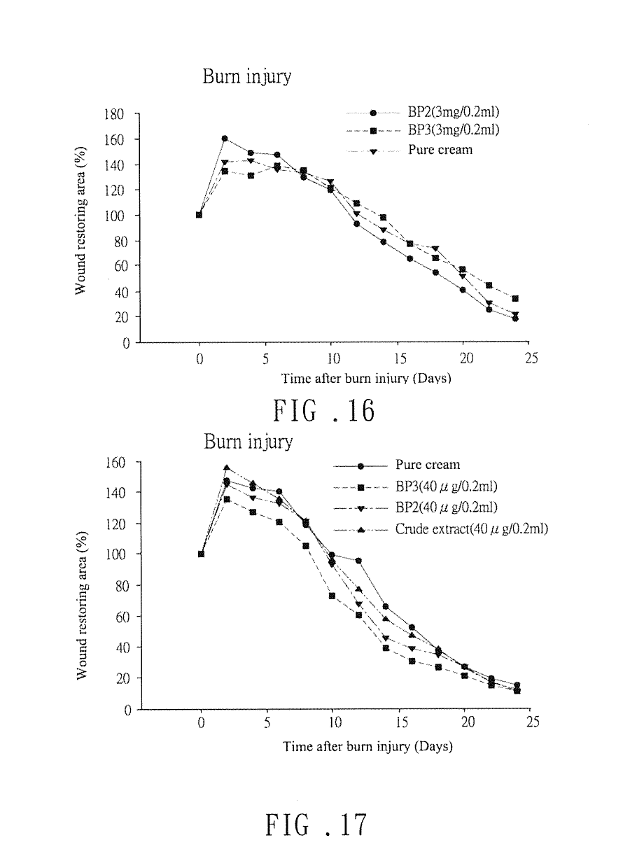

With reference to FIG. 16, a copper block at 175 degrees Celsius is placed on the back of the Wistar rat for ten seconds to respectively create four burned wounds. Afterward each wound is healed by 3 mg/0.2 ml BP2, BP3 and pure cream every day, and the relationship between days and wound recovery area % is verified after burning. BP3 inhibiting inflammation and controlling wound area is much better after providing medicine within eight days. BP2 provides better curative effect after eight days.

As shown in FIG. 17, 40 μg/0.2 ml BP2, BP3, crude extract, pure cream having different concentrations are additionally used to heal burned wounds. The relationship between days and wound recovery area % is verified after burning. BP3 and BP2 groups have the capability of speeding up the wound healing from the beginning of next day to the end of experiment.

C. Tissue Slice Test

1. Test Procedure

a. Pathologic Tissue Slice and HE Staining

After the Wistar rat that suffers injuries for specific days according to experimental design is performed with humanity sacrifice, PBS containing heparin (0.2 U/ml) at 4 degrees Celsius is injected into aorta until PBS carrying no color flows out of the vein. 4% paraformaldehyde at 4 degrees Celsius is injected to fasten it, and finally, the wounded area is carefully taken out through surgical blades and immersed into 10% Formalin fixing solution to reserve at 4 degrees Celsius, thereby fixing for several days.

Next, fixed tissue is performed to carry out dehydration and wax infiltration treatments. The skin tissue is performed with dehydration and wax infiltration by utilizing a tissue automatic processing system. Afterward, the tissue is embedded by a paraffin embedding machine into paraffin blocks. After performing tissue block section through a paraffin microtome, tissue slices are stained by using hematoxylin and eosin stain manner. While in completion, the slice is mounted by micro-mount, and a completed sample slice then is placed on an optical microscope for observation, the slice result is further photographed and recorded by the digital image capture system.

The operational flow path of pathologic tissue slide assay can be referred to the manner of Bayat (2005)12), and its whole is cited in reference information of the embodiment. Under 400× visual field, 20 regions are randomly selected from inside corium layers of each skin tissue, and the number of leukocyte with respect to neutrophils, macrophage and lymphocyte is performed with quantity assay. In addition, in order to evaluate the recovery of injured skin, 20 points are randomly and respectively selected from 3 portions of epidermis, dermis and striated muscle of each skin tissue sample, and three layers thickness then are respectively measured. Regarding the foregoing concern, experimenters that do not know animal grouping perform operating for the foregoing pathologic tissue slice assay.

b. Immunohistochemistry Staining:

The skin tissue is transposed in 30% sucrose solution at 4 degrees Celsius for overnight after fixing it for two hours via 4% para-formaldehyde. In order to reduce the difference of immunohistochemistry, the embodiment adopts the method revised from the previous research (Sung et al., 2003; Chen et al., 2008) 13,14) to perform operating so that each group of skin tissue is embedded in the same tissue block. The tissue blocks that are completed with embedding are performed with tissue slice at −30 degrees Celsius by using a freezing microtome. Each slice thickness is 5 μm. The frozen slice samples are placed into 4% para-formaldehyde for ten minutes after drying them at room temperature for one hour. Further, the samples are respectively covered by PBS containing antibodies (containing 0.01% Triton X-100 and 2% goat serum) at 4 degrees Celsius for overnight after they are reacted by 4% goat serum diluted by PBS at room temperature for one hour. Afterward Alexa Fluor 488-conjugated secondary monoclonal antibody having green fluorescence or rhodamine-conjugated secondary polyclonal antibody having red fluorescence respectively covers the slices to react at room temperature for one hour. Leica DM-6000B fluorescent microscope (Leica, Wetzlar, Germany) is used to observe the slice after using secondary polyclonal antibodies, and a SPOT Xplorer digital image capture system (Diagnostic Instruments, Inc., Sterling Heights, Mich., U.S.A.) is used to capture images. While observing green fluorescence, the laser wavelength of the fluorescent microscope is set at 488 nm. While observing red fluorescence image, the laser wavelength is set at 568 nm. A. 2.5 times of the objective lens is used to fix the same exposure time while capturing fluorescent images every time. The image size is retained at the same dimension under each experimental condition. Observers that do not know the situation use Image J software (National Institute of Health, Bethesda, Md. U.S.A.) to calculate immunological activity for each group when they are not notified the experimental content.

2. Test Result:

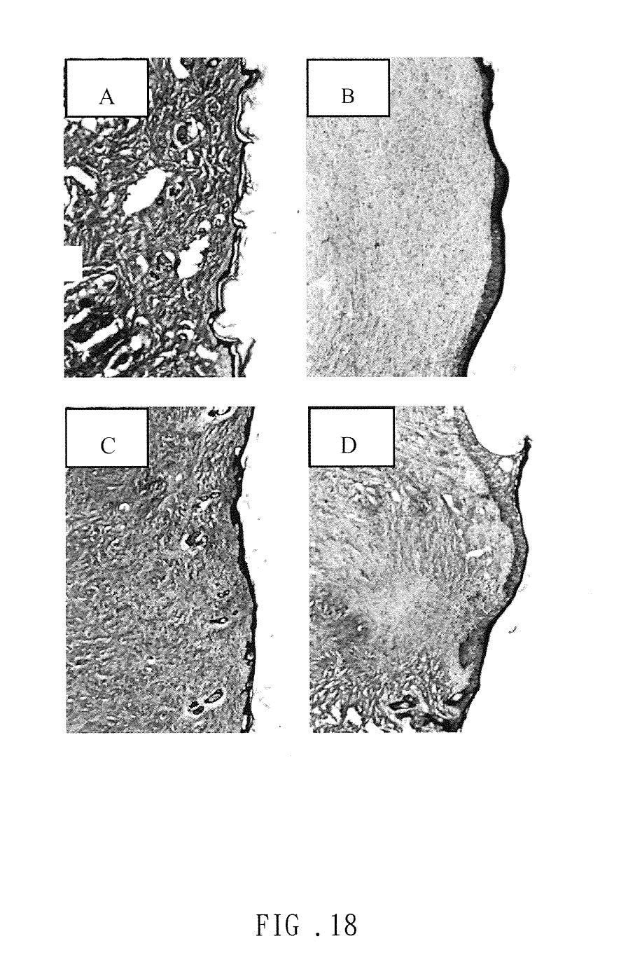

In FIG. 18, HE staining (5×) is used to discuss the burned skin tissue histopathological slides of the Wistar rat affected by crude extract. FIG. 18A is the control group, FIG. 18B is the burned group, FIG. 18C is the BP2 group, and FIG. 18D is the BP3 group. The concentration of the provided natural substance is 3 mg/0.2 ml. It can be seen that the lamina epithelialis of the burned group (figure B) and the healing group (figure C and figure D) is thickened relative to the control group (figure A), which is not injured, from HE photographing result. More particularly, the burned group (figure B) is thicker than the healing group (figure C). Relative to the burned group, the healing group's surface of the epidermis is smoother, granulation tissue can be found in the burned group and the healing group to show that burns would damage dermal tissues and induce inflammatory cell infiltration while the degree of infiltrating the healing group is lower than the burned group.

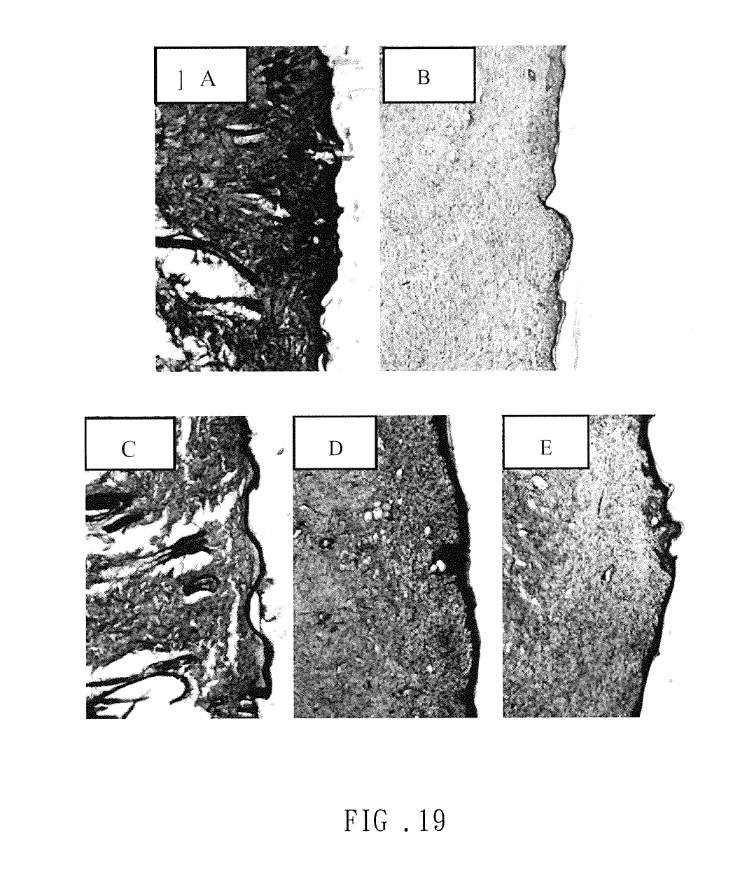

In FIG. 19, HE staining (5×) to discuss the burned skin tissue histopathological slides of the Wistar rat affected by crude extract. FIG. 19A is the control group, FIG. 19B is the burned group, FIG. 19C is the BP2 group, and FIG. 19D is the BP3 group, and FIG. 19E is the crude extract. The concentration of the provided natural substance is 40 μg/0.2 ml. It can be seen that numerous neutrophil are produced within lamina epithelialis of the burned group (figure B) and the healing group (figure E) relative to the control group (figure A), which is not injured, from HE photographing result. Neutrophil within lamina epithelialis of the healing group (figures C, D) is lesser, and hair follicles have already regenerated in the healing group (figure C).

Based upon the foregoing results, the burned wound can be recovered by BP2 and BP3 at acceleration, and healing effect at low concentration 40 μg/0.2 ml is much better than 3 mg/0.2 ml.

### TEST EXAMPLE 4

### Efficacy Test of Prototype Skincare Product from Briareum's Active Constituent

1. Skin Improvement Monitoring Performed by Skin Detector:

Aramo-TS multifunctional skin detector is used to perform skin efficacy evaluation. Inner sides of arms of ten subject persons are divided into regions to smear with test samples (blank-based agent, coral cream: BP2 (200 μg/ml) cream, BP3 (200 μg/ml) cream, coral crude extract (200 μl) cream) and are tested every week. After six weeks, the result is that the difference between background value and the smeared skin is analyzed by paired Student's T-Test. The active constituents of prototype skincare product from Briareum and the blank-based agent are summarized as the following table 2, wherein the difference between the blank-based agent and the coral cream only concerns the existence of any coral extract.

| Prototype Skincare Product Title | Coral Cream |
|---|---|
| Abbreviations | INCI |
| Content | Purified Water |
| | Hydroxyethyl Cellulose |
| | 1,3-Butylene Glycol |
| | Squalane |
| | Sodium Hyaluronate |
| | Glycerin |
| | Caprylic/Capric Triglyceride |
| | Sodium Polyacryloyldimethyl Taurate |
| | (And) Hydrogenated Polydecene |
| | (And) Trideceth-10 copolymer |
| | Vitamin B3: Niacinamide |
| | Vitamin E: Tocopheryl Acetate |
| | Methyl p-hydroxybenzoate (MP) |
| | Methylisothiazolinone (9.5%) |
| | Briareum excavatum extract: |
| | (BP2(200 μg/ml), |
| | BP3 (200 μg/ml) cream, and coral crude |
| | extract (200 μg/ml)) |

2. Test Result

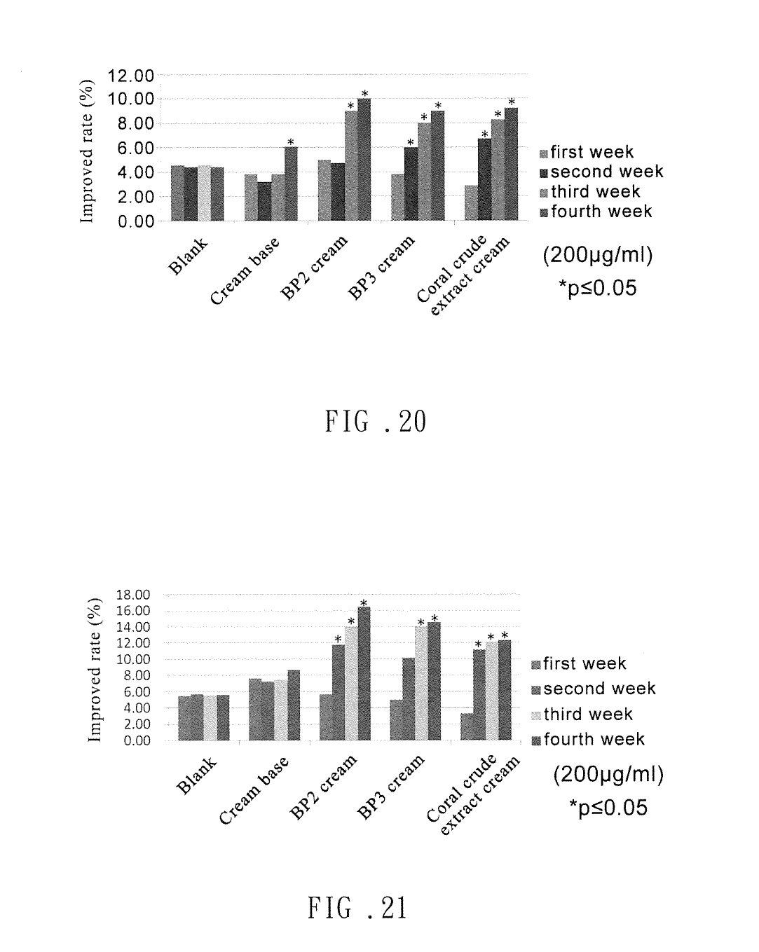

The result is shown in FIG. 20 and FIG. 21, the moisture retention and flexibility of skins of subject persons is obviously improved after using the prototype skincare product added with Briareum's active constituent for four weeks. More particularly, BP2 cream containing coral constituent has the best efficacy.

While the means of specific embodiments in the present invention has been described by reference drawings, numerous modifications and variations could be made thereto by those skilled in the art without departing from the scope and spirit of the invention set forth in the claims. The modifications and variations should in a range limited by the specification of the present invention.

What is claimed is:

1. A coral extract for whitening, moisture retention, elasticity improvement, anti-inflammation and wound healing of skin, the coral extract obtained by steps:
a. extracting dried Briaerum excavatum samples from an organic mixture to obtain an organic layer initial extract, wherein the organic mixture constitutes C₁-C₄ alcohol and C₁-C₄ chlorinated alkanes in equal proprotions; and
b. distributing and extracting the organic layer initial extract through water and low class ester to obtain a low class ester crude extract, the low class ester crude extract containing a 5-50% coral active constituent.

2. The coral extract of claim 1, wherein the step b of the low class ester extract is further purified by column chromatography, and the low class ester crude extract is eluted by eluent to obtain an eluted substance, wherein gradient elution of the eluent is provided by a first mixed solvent of hexane/ethyl acetate and a second mixed solvent of ethyl acetate/methanol.

3. The coral extract of claim 1, wherein the C₁-C₄ alcohol of the step a is methanol.

4. The coral extract of claim 1, wherein the C₁-C₄ chlorinated alkanes of the step a is methylene chloride.

5. The coral extract of claim I, wherein the low class ester of the step b is ethyl acetate.

6. The coral extract of claim 2, wherein a solvent gradient of hexane within the first mixed solvent is 0% to 99%.

7. The coral extract of claim 2, wherein a solvent gradient of ethyl acetate within the second mixed solvent is 0% to 90%.

8. The coral extract of claim 6, wherein the coral active constituent is obtained by eluting the low class ester crude extract through the first mixed solvent, wherein gradient elution of the first mixed solvent is that: hexane: ethyl acetate is fraction of 90:10 to 70:30.

9. The coral extract of claim 8, wherein the coral active constituent is 5 to 30% proportion of the low class ester crude extract after elution.

10. The coral extract of claim 1, wherein the coral active constituent is obtained by eluting the low class ester crude extract through the first mixed solvent, wherein gradient elution of the first mixed solvent is that: hexane: ethyl acetate is fraction of 60:40 to 50:50.

11. The coral extract of claim 10, wherein the coral active constituent is 20 to 50% proportion of the low class ester crude extract after elution.

12. An extracting method for coral extract of claim 1, comprising:
a. extracting dried Briareum excavatum samples through an organic mixture to obtain an organic layer initial extract, wherein the organic mixture contains C₁-C₄ alcohol and C₁-C₄ chlorinated alkanes in equal proprotions;
b. distributing and extracting the organic layer initial extract through water and low class ester to obtain a low class ester crude extract, the low class ester crude extract containing a 5-50% coral active constituent.

13. The extracting method of claim 12, wherein the low class ester crude extract of the step b is further purified by column chromatography, and the low class ester crude extract is eluted by eluent to obtain an eluted substance, wherein gradient elution of the eluent is provided by a first mixed solvent of hexane/ethyl acetate and a second mixed solvent of ethyl acetate/methanol.

14. A purpose of coral extract for imposing the coral extract of claim 1 on live skin in order to whiten skin.

15. A purpose of coral extract for imposing the coral extract of claim 1 on live skin in order to perform moisture retention.

16. A purpose of coral extract for imposing the coral extract of claim 1 on live skin in order to improve skin's elasticity.

17. A purpose of coral extract for imposing the coral extract of claim 1 on live skin in order to resist inflammation.

18. A purpose of coral extract for imposing the coral extract of claim 1 on live skin in order to heal wound.

19. A skincare product comprising the coral extract of claim 1, wherein the coral extract has 0.00001 to 10% weight percentage.

20. A skincare product comprising the coral extract of claim 2, wherein the coral extract has 0.00001 to 10% weight percentage.

21. A skincare product comprising the coral extract of claim 3, wherein the coral extract has 0.00001 to 10% weight percentage.

22. A skincare product comprising the coral extract of claim 4, wherein the coral extract has 0.00001 to 10% weight percentage.

23. A skincare product comprising the coral extract of claim 5, wherein the coral extract has 0.00001 to 10% weight percentage.

24. A skincare product comprising the coral extract of claim 6, wherein the coral extract has 0.00001 to 10% weight percentage.

25. A skincare product comprising the coral extract of claim 7, wherein the coral extract has 0.00001 to 10% weight percentage.

26. A skincare product comprising the coral extract of claim 8, wherein the coral extract has 0.00001 to 10% weight percentage.

27. A skincare product comprising the coral extract of claim 9, wherein the coral extract has 0.00001 to 10% weight percentage.

28. A skincare product comprising the coral extract of claim 10 wherein the coral extract has 0.00001 to 10% weight percentage.

29. A skincare product comprising the coral extract of claim 11, wherein the coral extract has 0.00001 to 10% weight percentage.
